(19) United States
(12) Patent Application Publication
(10) Pub. No.: US 2011/0262351 A1
(43) Pub. Date: Oct. 27, 2011

(54) FLUORESCENT SILICA NANOPARTICLE WITH RADIOACTIVE TAG AND THE DETECTING METHOD OF PET AND FLUORESCENT DUAL IMAGING USING THEREOF

(75) Inventors: DooSoo Chung, Seoul (KR); Keonwook Kang, Seoul (KR); Yonghyun Jeon, Seoul (KR); Younghwa Kim, Seoul (KR); Zeid A. Alothman, Riyadh (SA); Ahmed Yacine Hadj Ahmed, Riyadh (SA); Kihwan Choi, Seoul (KR); Abduullah M. Aimajid, Riyadh (SA); Jingyu Piao, Seoul (KR); Asma A. Alothman, Riyadh (SA); Bo Quan, Seoul (KR)

(73) Assignees: The Intellectual Property And Technology Licensing Program, Riyadh (SA); SNU R&DB Foundation, Gwanak-Gu, Seoul (KR)

(21) Appl. No.: 12/595,503

(22) PCT Filed: Sep. 9, 2009
(86) PCT No.: PCT/KR2009/005121

§ 371 Date: Jun. 27, 2011

(30) Foreign Application Priority Data
Sep. 9, 2008 (KR) 10-2008-0089014

(51) Int. Cl. A61K 51/04; A61P 43/00; C07F 7/18

(52) U.S. Cl. 424/1.65; 534/10

(57) ABSTRACT

The present invention relates to nuclear medicine using fluorescent silica nanoparticle and detecting method of optical dual imaging, and more particularly to radioisotope labeled fluorescent silica nanoparticles which are used for PET (positron emission tomography) and fluorescence detecting, and detecting method of PET and fluorescent dual imaging using thereof. Functionalized silica nanoparticles of this invention have promising potential as a role for organic lymphatic tracer in biomedical imaging such as pre- and intra-operative surgical guidance.

### TECHNICAL FIELD

The present invention relates to nuclear medicine using fluorescent silica nanoparticle and detecting method of optical dual imaging, and more particularly to radioisotope labeled fluorescent silica nanoparticles which are used for PET (positron emission tomography) and fluorescence detecting, and detecting method of PET and fluorescent dual imaging using thereof.

### BACKGROUND ART

Imaging technique capable of investigating location, extent, and transition of a tumor such as PET, MRI etc has been widely used. Although medical doctors can observe the extent of a tumor beforehand through said imaging, they can not observe the extent of a tumor during diagnosis or operation such as endoscopy, laparoscopy etc. So, they are hard to find transferred lymph node or happen not to remove the tumor completely.

Sentinel lymph node detection using gamma probe after injecting radioisotope is used clinically in breast cancer surgery. But it is hard to let the gamma probe approach along the route of transfer in abdominal cavity, because transfer direction in the case of abdominal cavity is too widespread in contrast to breast cancer. To compensate for this problem, dyes such as methylene blue are injected together, but the molecular weight too small to stay in lymph node.

Identifying sentinel lymph node through the monitor during operation using quantum dot injected to the pig was successful. But the practical use is restricted because quantum dot use the Cadmium (Cd) which is forbidden to use to human body.

Sentinel lymph node detection based on the use of radiolabeled colloid nanoparticles combined with blue dye during surgery in early breast cancer has become a standard means of reducing the extent of surgical exploration and post-operative morbidity (Radovanovic Z, Golubovic A, Plzak A, Stojiljkovic B, Radovanovic D., Eur J Surg Oncol 2004; 30:913-7; Rodier J F, Velten M, Wilt M, Martel P, Ferron G, Vaini-Elies V, et al., J Clin Oncol 2007; 25:3664-9). Moreover, sentinel node detection has now been adopted for other types of cancers (Roberts A A, Cochran A J., J Surg Oncol 2004; 85:152-61; Aikou T, Kitagawa Y, Kitajima M, Uenosono Y, Bilchik A J, Martinez S R, et al., Cancer Metastasis Rev 2006; 25:269-77). Although the amount of radioactivity used for sentinel node detection is low and generally considered safe, general concern of using radioisotope has been still aroused in the nursing and pathologic staff (Nejc D, Wrzesien M, Piekarski J, Olszewski J, Pluta P, Kusmierek J, et al., Eur J Surg Oncol 2006; 32:133-8). Accordingly, the uses of various non-radioactive materials, such as, fluorophore dyes and nanoparticles, have been investigated in the context of sentinel node detection (Table 1). However, the low molecular weights of fluorophore dyes mean that their residence times at sentinel nodes are limited, and thus, researchers have been trying to develop new materials for this purpose. Quantum dots (QDs) and macromolecular MRI contrast materials in combination with in vivo imaging systems have been used to locate sentinel lymph nodes in living organisms with high sensitivity and resolution. However, despite their potential benefits, the practical applications of quantum dots are limited by poor bio-compatibility and potential toxicity (Hardman R., Environ Health Perspect 2006; 114:165-72; Zhang T, Stilwell J L, Gerion D, Ding L, Elboudwarej O, Cooke P A, et al., Nano Lett 2006; 6:800-8).

TABLE 1
Studies conducted on sentinel lymph node detection using
nanoparticles and dyes

| Year | Authors | Objects | Used material |
|---|---|---|---|
| 2008 | Sevick-Muraca | Human | ICG(Sevick-Muraca E M, Sharma R; |
| | et al. | | Rasmussen J C, Marshall M V, |
| | | | Wendt J A, Pham H Q, et al., Radiology |
| | | | 2008; 246: 734-41) |
| 2007 | Kobayashi | Nude | Qdot 565, 605, 655, 705, and 800 |
| | et al. | mouse | (Kobayashi H, Hama Y, Koyama Y, |
| | | | Barrett T, Regino C A, Urano Y, et al., |
| | | | Nano Lett 2007; 7: 1711-6) |
| 2004 | Kim et al. | Swine | Quantum dot 840(Kim S, Lim Y T, |
| | | | Soltesz E G, De Grand A M, Lee J, |
| | | | Nakayama A, et al., NatBiotechnol |
| | | | 2004; 22: 93-7) |
| 2005 | Pelosi et al. | Human | ^{99m}Tc-labeledalbumin nanocolloid and |
| | | | blue blue dye(Pelosi E, Ala A, Bello M, |
| | | | Douroukas A, Migliaretti G, Berardengo |
| | | | E, et al., Eur J Nucl Med Mol Imaging |
| | | | 2005; 32: 937-42) |
| 2003 | Josephson | Nude | Cy5.5(Josephson L, Mahmood U, |
| | et al. | mouse | Wunderbaldinger P, Tang Y, Weissleder |
| | | | R., Mol Imaging 2003; 2: 18-23) |
| 2001 | Simmons et al. | Human | Methylene blue dye(Simmons R M, |
| | | | Smith S M, Osborne M P., Breast J |
| | | | 2001; 7: 181-3) |
| 2000 | Rety et al. | Rat | Superparamagnetic nanoparticle fer- |
| | | | umoxtran(Rety F, Clement O, Siauve N, |
| | | | Cuenod C A, Carnot F, Sich M, et al., J |
| | | | Magn Reson Imaging 2000; 12: 734-9) |
| 1996 | Karakousis | Human | Rosaniline dye(Karakousis C P, Velez |
| | et al. | | A F, Spellman J E, Jr., Scarozza J., |
| | | | Eur J Surg Oncol 1996; 22: 271-5) |
| 1993 | Alex and Krag | Cat | ^{99m}Tc sulfur colloid(Alex J C, Krag D N., |
| | | | Surg Oncol 1993; 2: 137-43) |
| | Alex et al. | Human | ^{99m}Tc sulfur colloid(Alex J C, Weaver |
| | | | D L, Fairbank J T, Rankin B S, Krag |
| | | | D N., Surg Oncol 1993; 2: 303-8) |
| 1980 | Hirsch et al. | Human | Isosulfan blue dye(Hirsch J I., Am J Hosp |
| | | | Pharm 1980; 37: 1182-3) |

ICG; Indocyanine Green,
Qdot; Quantum dot,
^{99m}Tc; Technetium-99m

Functionalized silica nanoparticles can be made by incorporating fluorescent dye molecules within the silica matrix, and can be easily conjugated with many other bio-molecules (Yoon T J, Yu K N, Kim E, Kim J S, Kim B G, Yun S H, et al., Small 2006; 2:209-15; Wang J, Liu G, Lin Y., Small 2006; 2:1134-8; Bank T K, Sahu B, Swain V., Parasitol Res 2008; 103:253-8; Yoon T J, Kim J S, Kim B G, Yu K N, Cho M H, Lee J K., Angew Chem Int Ed Eng12005; 44:1068-71).

Furthermore, Kim et al. investigated the toxicity and tissue distribution of SiO₂ nanoparticles in mice, and found that they had no significant long-term toxicity under the experimental conditions used (Kim J S, Yoon T J, Yu K N, Kim B G, Park S J, Kim H W, et al., Toxicol Sci 2006; 89:338-47). However, although several studies have concluded that functionalized silica nanoparticles can be applied in various biological and medical areas, functionalized silica nanoparticles was not applied to in vivo animal study using optical imaging.

But these functionalized silica nanoparticles have not been applied to the in vivo animal research in the nuclear medicine and optical imaging. Also, in conventional examination of sentinel lymph node, we can not confirm radioisotpe nanoparticle during operation, and dying material was so small that it pass the sentinel lymph node. Also, conventional nano fluorescent quantum dots use the Cd, so, it is hard to apply those to human body.

The inventors of the present invention tried obtaining optical imaging of living things using toxicity free material, we knew that silica nanoparticle is harmless to human and is functionalized to fluoresce and also can be used to detect sentinel lymph node. We complete this invention by succeeding to obtain PET/fluorescence dual imaging of sentinel lymph node by introducing radioisotope to silica nanoparticles doped with fluorescent dye such as rhodamine, indocyanine green.

### DISCLOSURE OF INVENTION

### Technical Problem

The object of this invention is to provide nanoparticle useful to detect PET/fluorescence dual imaging and detecting method thereof, particularly, radioisotope labeled fluorescent silica nanoparticles and PET/fluorescence dual imaging of sentinel lymph node using them.

### Technical Solution

In accordance with an aspect of the present invention, the present invention can be accomplished by the provision of radioisotope labeled fluorescent silica nanoparticles which are used for PET (positron emission tomography) and fluorescence detecting.

Also, the present invention provides detecting method of PET and fluorescent dual imaging using radioisotope labeled fluorescent silica nanoparticles.

The present invention describes in detail herein.

In radioisotope labeled fluorescent silica nanoparticles which are used for PET (positron emission tomography) and fluorescence detecting, it is preferable that the fluorescent dye doped in said nanoparticles is dye for near infrared ray, and said radioisotope is ⁶⁸Ga or ¹³¹I. And it is desirable that said radioisotope labeled fluorescent silica nanoparticles are ⁶⁸Ga-NOTA-silica nanoparticles.

Also, the present invention provides detecting method of PET and fluorescent dual imaging using radioisotope labeled fluorescent silica nanoparticles. More particularly, the present invention is detecting method of PET and fluorescent dual imaging comprising the steps of [symbol] ) manufacturing radioisotope labeled fluorescent silica nanoparticles; and [symbol] ) obtaining PET/fluorescent in vivo imaging of lymph node or tracing bio-distribution using said nanoparticles. It is desirable that fluorescent material of said nanoparticles is TMR or ICG, and said radioisotope labeled fluorescent silica nanoparticle is Ga-68 labeled NOTA-silica nanoparticle, and said lymph node is sentinel lymph node.

Fluorescent silica nanoparticle material is possible to be applied to clinic because it affects the human body insignificantly, and the full imaging of human body can be obtained when radioisotope for PET is labeled to it. The present invention can provide PET and fluorescent dual imaging of sentinel lymph node using radioisotope labeled fluorescent silica nanoparticles.

Also, the present invention is the manufacturing method of radioisotope labeled fluorescent silica nanoparticles comprising the steps of i) making the fluorescent silica nanoparticles by doping fluorescent dye in interior of silica; ii) modifying the surface of said silica nanoparticles in order to introducing biomolecules or ligands; and iii) coupling the radioisotope for PET to said modified silica nanoparticles.

It is desirable that the step of modifying the surface comprises introduction of amine group, and NOTA or DOTA group is introduced to amine group in order to introduce radioisotope more easily.

We describe the present invention step by step in below

\textcircled{1} The Preparation of Radioisotope Labeled Fluorescent Silica Nanoparticles

For nano material for nuclear medicine (PET in narrow category)/optics (fluorescence in narrow category) dual imaging of sentinel lymph node, we mix radioisotope with nano material precursor and shake them. We synthesize silica nanoparticles doped with fluorescent dye such as TMR, ICG, and then Ga-68 labeled NOTA-silica nanoparticle.

First, we synthesize silica nanoparticles doped with fluorescent dye. Recently, luminous material in nano size is attractive in detection in biological sample. Specially, silica nanoparticles are more attractive because of high stability, living things adaptation, and radiance intensified character, and they can be synthesized by reverse micro emulsion or Stober method, and they have a big fluorescent signal because there are thousands of or ten thousands of fluorescent dyes in silica inner layer. Also, abrupt photobleaching by oxygen is prohibited because dyes and solution are separated by silica layer and it shows photostability. Besides, the surface of silica nanoparticle is easy to introduce several biomolecule or ligands. The inventors synthesized silica nanoparticle doped with tetramethylrhodamine, tris(2,2-bipyridyl)-dichlororuthenium(II) hexahydrate (Ru(bpy)₃²⁺), etc, and FIG. 3 is the TEM image of it.

We examine the influence of the size of nanoparticle, the kind of fluorescent dyes, and the concentration of nanoparticle for lymph node imaging. We control the size of nanoparticle by controlling the ratio of water and surfactant because the size of produced nanoparticle is affected by the size of micro emulsion water drop, and generally the size of nanoparticle become small when the ratio of surfactant become high. We need to control the size of nanoparticle properly because it pass the lymph node when its size too small, and it moves too slowly when its size too big. Silica nanoparticle is a good material because silica nanoparticle can be made as a size from several nm to hundreds nm. The dye for near infrared ray is desirable to be doped to nanoparticles. Infrared ray is suitable for living organism imaging because of high permeability to living organism. In the present invention, we raise the efficiency of imaging by selecting the proper dye into silica nanoparticles. We control the concentration of silica nanoparticles. It is possible to lower the concentration because the signal of nanoparticle is strong compared with ordinary dye molecule, generally.

Next, radioisotope is labeled to silica nanoparticles doped with fluorescent dyes. There are about 20 kinds of radioisotope with useful positron decay and proper half life theoretically for PET, but the use of them is restricted by several practical reasons, and ⁶⁸Ga is used typically. The half life of ⁶⁸Ga is relatively short as 68 minute, and it is labeled to cationic chelate such as NOTA (1,4,7-triazacyclononanetriacetic acid) in the condition of Ga+3. Of course, DOTA (1,4,7,10-tetraazacyclododecanetetraacetic acid) as a cationic chelate can be used. Especially, ⁶⁸Ga is usable in the needed place through ⁶⁸Ge/⁶⁸Ga generator without cyclotron, and the half life of ⁶⁸Ge is about 270 days, so the generator can be used consistently about 1 year without replacement.

Cationic chelate such as NOTA is usually used in labeling peptide or protein, and has —NCS group attachable to —NH₂ group of peptide or protein. So, besides peptide or protein, any other compounds with —NH₂ group are applicable, particularly, in the present invention, we coupled NOTA with modified silica nano particle introduced —NH₂ group at the surface.

Labeled ⁶⁸Ga-NOTA is much stable and was stable in 6 M HNO₃ for over 6 hours. So, ⁶⁸Ga labeled silica nano particles doped with fluorescent dyes are stable.

We introduce —NH₂ group at the surface of silica nanoparticle before the introduction of ⁶⁸Ga labeling, and then coupled NCS-NOTA (2-(4′-isocyanatobenzyl)-1,4,7-triazacyclononanetriacetic acid). The NOTA-silica nanoparticles react with ⁶⁸GaCl₃ solution eluted from ⁶⁸Ge/⁶⁸Ga generator and then ⁶⁸Ga-NOTA-silica nanoparticles are synthesized.

\textcircled{2} PET/Fluorescence Dual Imaging of Sentinel Lymph Node

We decide optimized dosage, size, etc through the model of breast cancer sentinel lymph node by hypodermic injection, and then apply it to laparoscopy simulation model of sentinel lymph node such as stomach cancer, colon cancer, etc. ⁶⁸Ga-NOTA-silica nanoparticles are injected to hypodermis of mouse, and then PET and Fluorescent imaging is obtained.

First, we get the fluorescent imaging of living organism using silica nanoparticles. For this, nude mouse without fur is used. Cut the portion showing proper fluorescence and observe through fluorescent microscope, and confirm the lymph node with H&E dyeing. Measure the fluorescence remained in body through full photography of mouse after cutting the lymph node.

Second, we get the PET and Fluorescent imaging after injecting ⁶⁸Ga-NOTA-silica nanoparticles to the hypodermis of mouse. Get the full photography of mouse according to the time using the PET/CT after injecting proper silica nanoparticle to nude mouse, and when sentinel lymph node turn out, get the fluorescent imaging from it. After cutting the portion showing fluorescence, and measure the amount of radiation and observe it through fluorescent microscope. Measure the amount of radiation remained in body through full PET of mouse after cutting the lymph node.

\textcircled{3} Tracing Bio-Distribution of Radioisotope Labeled Fluorescent Silica Nanoparticles

In order to confirm safety of injecting nano material to living organism, trace the staying time, evacuation route, and accumulated internal organs. Trace bio-distribution and evacuation route for over 2 weeks after injecting I-131 labeled silica nanoparticles.

### Advantageous Effects

Functionalized silica nanoparticles of this invention have promising potential as a role for sentinel lymphatic tracer through PET and fluorescent dual imaging in surgical guidance.

### BRIEF DESCRIPTION OF DRAWINGS

FIG. 1 shows the procedure to obtain the imaging using radioisotope and fluorescence schematically.

FIG. 2 shows the procedure to make silica nanoparticle using reverse micro emulsion method schematically.

FIG. 3 is TEM image for silica nano particle.

FIG. 4 shows ⁶⁸Ga labeling of peptide or protein using cationic chelate.

FIG. 5 shows the procedure of —NH₂ group introduction using 3-aminopropyltrimethoxysilane.

FIG. 6 shows the procedure of synthesis of NOTA-silica nanoparticle for ⁶⁸Ga labeling.

FIGS. 7, 8 is in vitro fluorescent imaging (FIG. 7) of the mouse after hypodermic injection of silica nanoparticle with various concentration, and the quantified graph (F 8) of said imaging.

FIGS. 9, 10 is in vivo biodistribution (FIG. 9) of nano silica with biooptic imaging equipment in a day after manufactured nano silica was injected into right fore foot pad, and the imaging after extraction of all organs (FIGS. 9,10).

FIG. 11 is Ex vivo validation of RITC-SiO₂ nanoparticles. A is Ex vivo fluorescent image of extracted lymph nodes. In vivo fluorescent images were acquired after skin removal at 30 min post RITC-SiO₂ injections to locate sentinel lymph nodes. After in vivo whole body imaging acquisition, mice were sacrificed and eight lymph nodes were extracted to detect specific uptakes in axillary and brachial lymph nodes. B is Ex vivo fluorescence imaging of organs. Animals were sacrificed and all organs were removed and imaged at 30 min post RITC-SiO₂ injection. ALN; axillary lymph node, IN; inguinal lymph node, SN; sciatic lymph node, BLN; brachial lymph node, SCN; superficial cervical lymph node. All images were acquired under the same experimental conditions.

FIG. 12 is biodistribution of ⁶⁸Ga-NOTA-RITC-SiO₂ nanoparticles in nude mice. Mice were sacrificed 30 min after injecting 50 mCi of ⁶⁸Ga-NOTA-RITC-SiO₂s.c. into the right fore foot-pads. Organs were then removed and weighed, and radioactivities were measured. ALN; axillary lymph node, IN; inguinal lymph node, SN; sciatic lymph node, BLN; brachial lymph node, SCN; superficial cervical lymph node. Data are expressed as % ID/g of tissue. n=5 mice

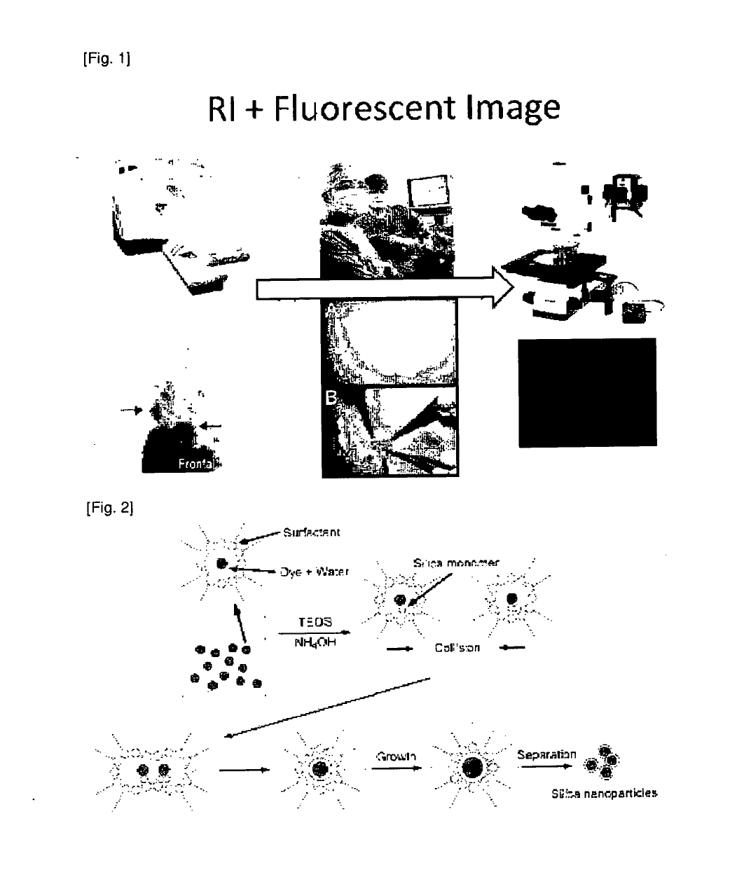

### BEST MODE FOR CARRYING OUT THE INVENTION

Exemplary embodiments of the present invention will be described in detail below. The present invention may, however, be embodied in many different forms and should not be construed as limited to the exemplary embodiments set forth herein. Rather, these exemplary embodiments are provided so that this disclosure is thorough and sufficient description of the present invention, and will fully convey the spirit of the invention to those skilled in the art.

### Embodiment 1

### Animals and Chemicals

<1-1> Animals

Specific pathogen-free six-week-old female BALB/c nude mice were obtained from SLC Inc. (Japan). All animal experiments were performed after receiving approval from the Institutional Animal Care and Use Committee (IACUC) of the Clinical Research Institute at Seoul National University Hospital. In addition, the National Research Council (NRC) guidelines for the care and use of laboratory animals (revised 1996) were observed throughout.

<1-2> Chemicals

Rhodamine β isothiocyanate (RITC), 3-(aminopropyl)triethoxysilane (APTS), and phosphate buffered saline (PBS, pH 7.4) were obtained from Sigma (St. Louis, Mo.). Tetraethyl orthosilicate (TEOS), and 29 wt % aqueous ammonia solution were from Aldrich (Milwaukee, Wis.). 2-[Methoxy(polyethylenoxy)propyl] trimethoxysilane (PEG-silane, 90%) were from Gelest (Morrisville, Pa.).

### Embodiment 2

### The Preparation of Radioisotope Labeled Fluorescent Silica Nanoparticles

<2-1> The Preparation of Silica Nanoparticles Doped with Fluorescent Dyes

Silica nanoparticles were made by reverse micro emulsion method (FIG. 2). FIG. 3 is the TEM image of it. Silica nanoparticles doped with fluorescent dyes is manufactured by introducing RITC to said silica nanoparticles.

<2-2> The Preparation of Ga-68 Labeled NOTA-Silica Nanoparticles for PET

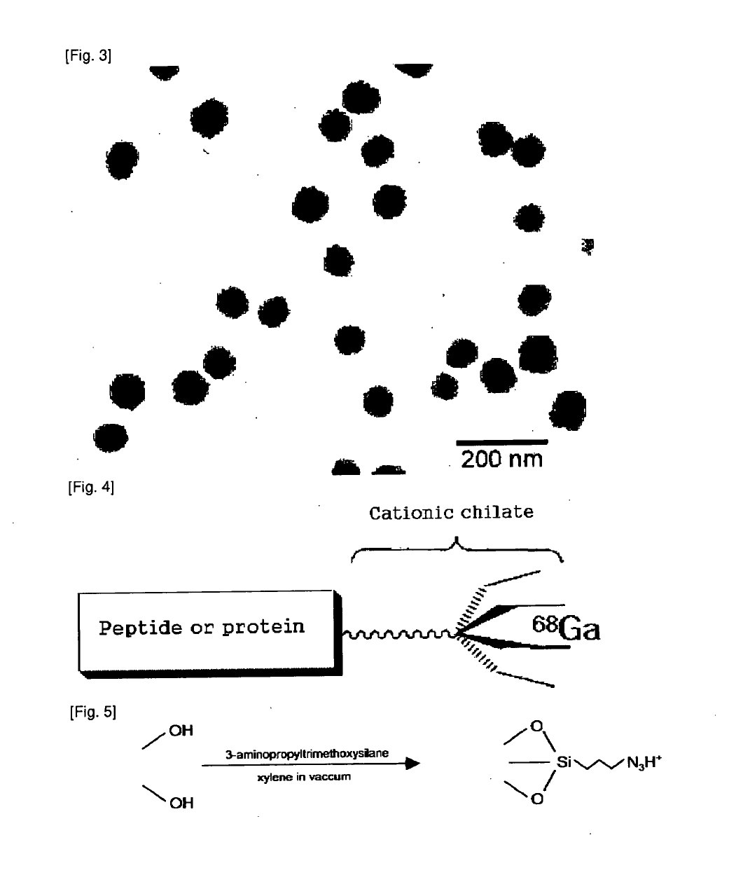

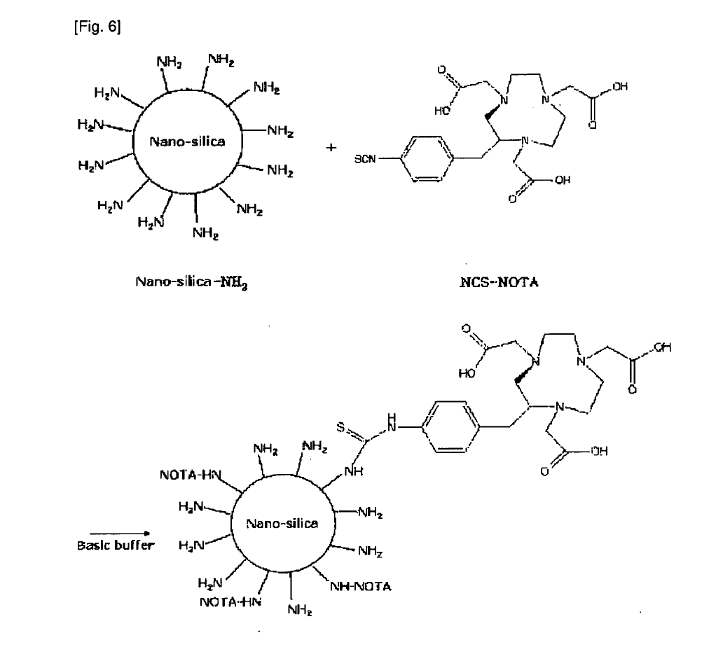

Labeled ⁶⁸Ga-NOTA is much stable and was stable in 6M HNO₃ for over 6 hours. So, ⁶⁸Ga labeled silica nano particles doped with fluorescent dyes are stable. We introduce —NH₂ group at the surface of silica nanoparticle before the introduction of ⁶⁸Ga labeling, and then coupled NCS-NOTA (2-(4′-isocyanatobenzyl)-1,4,7-triazacyclononanetriacetic acid). The NOTA-silica nanoparticles react with ⁶⁸GaCl₃ solution eluted from ⁶⁸Ge/⁶⁸Ga generator and then ⁶⁸ Ga-NOTA-silica nanoparticles are synthesized (FIG. 4 to FIG. 6).

In the concrete, To the sodium carbonate solution (0.2 M, 1 mL), RITC-SiO₂ nanoparticles solution (100 mL) and 2-(4′-isothiocyanatobenzyl)-1,4,7-triazacyclononane-1,4,7-triacetic acid (p-SCN-Bn-NOTA, 2.0 mg, 3.6 nmol) were added. The mixture was stirred for 12 h at room temperature and centrifuged to remove supernatant, and washed with ethanol (1 mL) and water (1 mL), successively. The orange colored precipitate was re-dispersed in water (1 mL) and kept at −20° C. RITC-SiO₂ nanoparticles-SCN-NOTA solution (100 mL) and ⁶⁸GaCl₃ solution (287 MBq, 900 mL), which was freshly eluted from ⁶⁸Ge—⁶⁸Ga generator, were mixed and sodium phosphate solution (0.5 M, 220 mL) was added to adjust pH 5. The mixture was mixed and kept at 90° C. for 20 min. After the reaction, the reaction mixture was centrifuged and washed with de-ionized water (1 mL), and the precipitate was re-dispersed in water (1 mL) before injection. The radiochemical yield and radiochemical purity were checked by ITLC-SG (eluent: 0.1 M sodium carbonate or 0.1 M citric acid solution). The R_{f} value of ⁶⁸Ga-NOTA-SiO₂ nanoparticles was 0.1 with both eluents, and that of free ⁶⁸Ga was 0.1 using 0.1 M sodium carbonate solution and 1.0 using 0.1 M citric acid solution. The radiochemical yield was over 95% and radiochemical purity was over 99% after the purification.

### Embodiment 3

### PET/fluorescence dual imaging of sentinel lymph node

<3-1> Fluorescence Imaging

We use nude mouse without fur in order to get the full fluorescent imaging. In order to confirm the optimized dosages, kinds (Tetramethylrhodamine-5, Indocyanine green etc.) and size (10-100 nm) of fluorescent materials, we get the full imaging without radioisotope labeling according to the time using Xenogen IVIS 100 under 2% isoflurane gas anesthesia after injecting to hypodermis of nude mouse. We confirm lymph node after cutting the portion showing fluorescence, we take a picture using Xenogen IVIS 100 and observe through fluorescent microscope, and confirm the lymph node with H&E dyeing. We measure the fluorescence remained in body through full photography of mouse after cutting the lymph node.

Fluorescence images were obtained using a Maestro In Vivo Imaging System (CRi Inc., Woburn, Mass.) for data acquisition and analysis. Before imaging, mice were anesthetized i.p. with a solution containing 8 mg/mL ketamine (Ketalar, Panpharma, Fougeres, France) and 0.8 mg/mL xylazine (Rompun, Bayer Pharma, Puteaux, France) at 0.01 mL/g of body weight. RITC-SiO₂ nanoparticles (40 μg/40 μl) were injected s.c. into the right fore foot-pads of nude mice. Fluorescence measurements were performed at 5 min after foot-pad injections. In vivo fluorescence Measurements were taken on top of ALNs (axillary lymph nodes) after skin removal.

In all cases, optical image sets were acquired using a green filter set (a band-pass filter from 503 to 555 nm and a long-pass filter of 580 nm. which were used for excitation and emission, respectively) to acquire one complete image cube. The tunable filter was automatically increased in 10-nm increments from 550 to 800 nm. A camera was used to capture images at each wavelength using a constant exposure.

We get the in vivo imaging after subcutaneous injection of fluorescent silica nanoparticle into dorsal region. We get the full imaging using IVIS100 (Fluorescent/bioluminescence imaging machine) after injecting fluorescent silica nanoparticle into footpad.

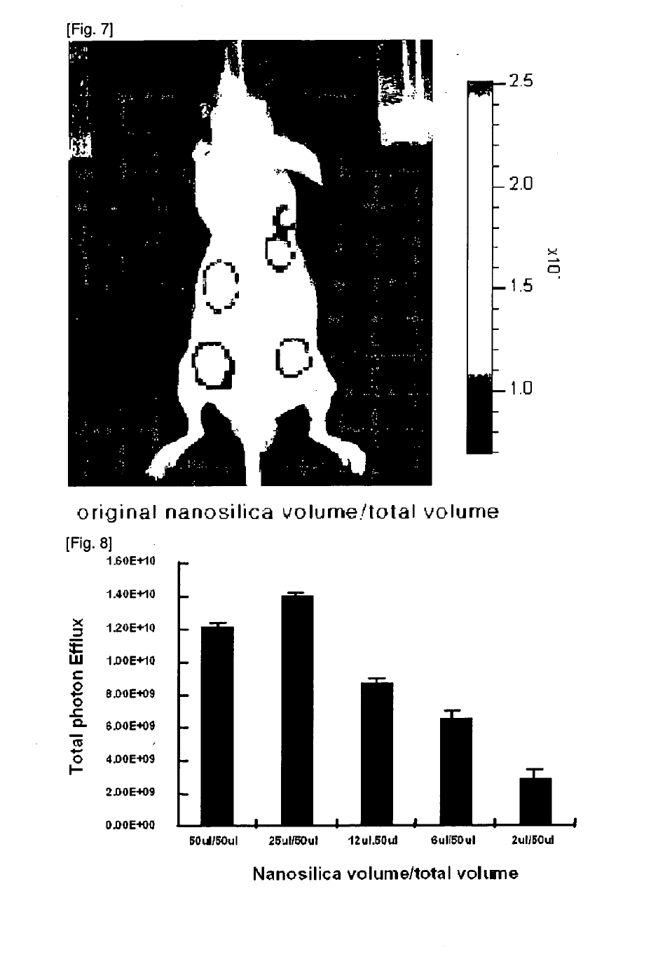

In the concrete, We get the full imaging using IVIS100 after subcutaneous injection of silica nanoparticle 2, 6, 12, 25, 50 μl/50 μl in PBS (FIG. 7). Quantitative analysis is carried out using the imaging around injection area after getting full imaging (FIG. 8). It is confirmed that imaging in injection area increases in proportion to volume of silica nanoparticle.

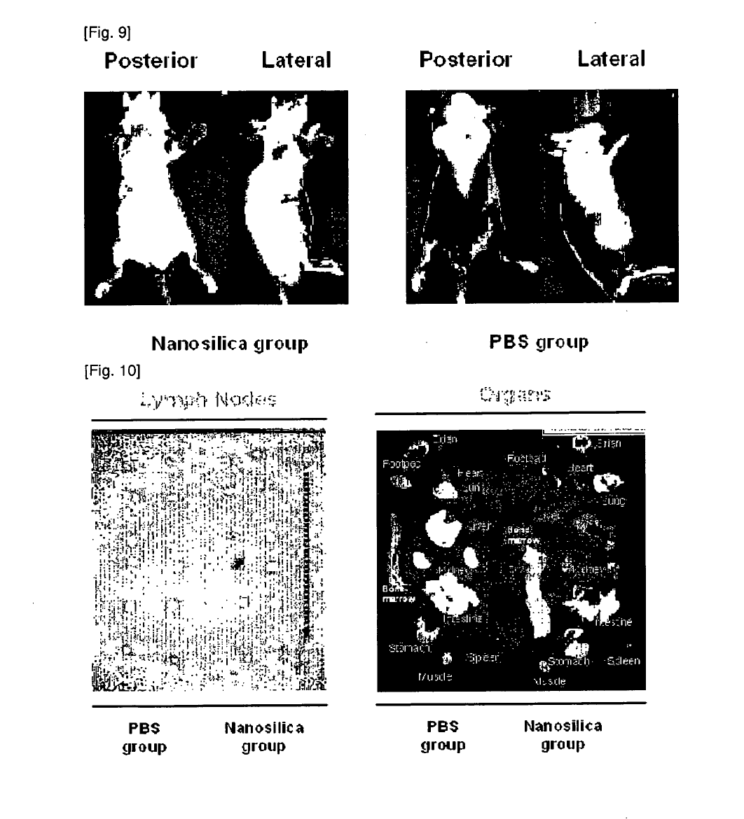

Also, we get the in vivo imaging after subcutaneous injection of fluorescent silica nanoparticle into footpad. In the concrete, we photograph distribution of silica nanoparticle using IVIS100 after injection of silica nanoparticle 50 μl into footpad (FIG. 9). Also, we photograph after the removal of organs (FIG. 10). As a result, we get the strong fluorescent imaging from injection area of footpad, and get the fluorescent imaging from draining lymph nodes.

<3-2> PET/Fluorescence Dual Imaging after Injecting ⁶⁸Ga-NOTA-Silica Nanoparticles Into the Hypodermis of Mouse

We get the full photography of mouse according to the time using the PET/CT after injecting proper silica nanoparticle to the hypodermis of nude mouse. When sentinel lymph node turned out, we get the fluorescent imaging from it using Xenogen IVIS 100. After cutting the portion showing fluorescence, we photograph the dissected organs with Xenogen IVIS 100, and measure the amount of radiation using gamma counter and observe it through fluorescent microscope. We measure the amount of radiation remained in body through full PET of mouse after cutting the lymph node.

### Embodiment 4

### Tracing Bio-Distribution of Radioisotope Labeled Fluorescent Silica Nanoparticles

We trace bio-distribution and evacuation route for over 2 weeks after injecting I-131 labeled silica nanoparticles. In the concrete, in order to confirm safety of injecting nano material to living organism, we injected I-131 (half life of 8 days) labeled silica nanoparticles into the hypodermis of mouse to estimate the staying time, evacuation route, and accumulated internal organs. After photographing full imaging with gamma camera with pinhole collimator, we removed injection part and sentinel lymph node part which is assumed as sentinel lymph node and stitched it. We measure the amount of radiation of removed organs with gamma counter. After assuming the bio-distribution and evacuation route using gamma camera imaging and full fluorescent imaging according to the date, we measure the amount of remained nano material by organs through estimating the amount of radiation with gamma counter and fluorescence with fluorescent microscope by extracting the liver, the spleen, the lungs, and the heart etc by organs.

We examined bio-distribution using nanoparticle manufactured in embodiment 2-2. Immunocompetent mice (n=5) were sacrificed 30 min after administering ⁶⁸Ga-NOTA-RITC-SiO₂ (50 mCi/50 ml) to right fore foot-pads. Organs were removed, weighed, and counted for radioactivity using a gamma counter. Results are expressed as percentages of doses injected per gram of tissue (% ID/g). As a result, it is confirmed that plenty of radioisotope are absorbed around draining lymph nodes.

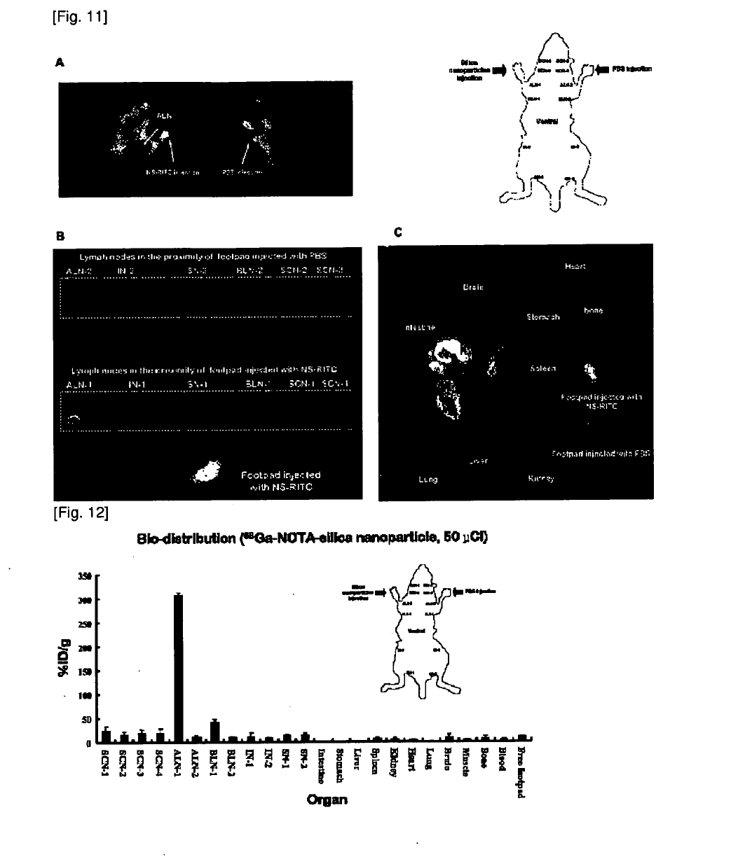

Mice were injected with silica nanoparticles and sacrificed 30 min post-injection. All organs including lymph nodes were removed and imaged. Except for three organs (axillary lymph node, brachial lymph node, and injection foot-pad), fluorescence signals were not detected in the other tested organs (FIG. 11). Also, we examined bio-distribution of ⁶⁸Ga-NOTA-RITC-SiO₂ in nude mouse. The % ID/g of axillary lymph node, brachial lymph node around foot-pad treated with ⁶⁸Ga-NOTA-RITC-SiO₂ nanoparticle is respectively 308.3±3.4 and 41.5±6.1 (FIG. 12). The radioactivity of ⁶⁸Ga is not found in any other organs significantly (for example in liver, lungs, brain, spleen and kidney). FIG. 11 and FIG. 12 prove that the bio-distributions of RITC-SiO₂ and ⁶⁸Ga-NOTA-RITC-SiO₂ are similar.

1. Radioisotope labeled fluorescent silica nanoparticles which are used for PET (positron emission tomography) and fluorescence detecting.

2. Radioisotope labeled fluorescent silica nanoparticles of claim 1, wherein the fluorescent dye doped in said nanoparticles is dye for near infrared ray.

3. Radioisotope labeled fluorescent silica nanoparticles of claim 1, wherein said radioisotope is ⁶⁸Ga or ¹³¹I.

4. Radioisotope labeled fluorescent silica nanoparticles of claim 3, wherein said nanoparticles is ⁶⁸Ga-NOTA-silica nanoparticles.

5. The detecting method of PET and fluorescent dual imaging using radioisotope labeled fluorescent silica nanoparticles according to any one of claim 1 to claim 4.

6. The detecting method of PET and fluorescent dual imaging of claim 5, comprising the steps of: [symbol]
) manufacturing radioisotope labeled fluorescent silica nanoparticles; and [symbol] ) obtaining PET/fluorescent in vivo imaging of lymph node or tracing bio-distribution using said nanoparticles.

7. The detecting method of PET and fluorescent dual imaging of claim 6, wherein fluorescent material of said nanoparticles is TMR or ICG.

8. The detecting method of PET and fluorescent dual-imaging of claim 6, wherein said radioisotope labeled fluorescent silica nanoparticle is Ga-68 labeled NOTA-silica nanoparticle.

9. The detecting method of PET and fluorescent dual imaging of claim 6, wherein said lymph node is sentinel lymph node.

10. The manufacturing method of radioisotope labeled fluorescent silica nanoparticles comprising the steps of:
i) making the fluorescent silica nanoparticles by doping fluorescent dye in interior of silica;
ii) modifying the surface of said silica nanoparticles in order to introducing biomolecules or ligands; and
iii) coupling the radioisotope for PET to said modified silica nanoparticles.

11. The manufacturing method of radioisotope labeled fluorescent silica nanoparticles of claim 10, wherein the step of modifying the surface comprises introduction of amine group.

12. The manufacturing method of radioisotope labeled fluorescent silica nanoparticles of claim 11, wherein NOTA or DOTA group is introduced to amine group.
